(19) United States
(12) Patent Application Publication
(10) Pub. No.: US 2010/0010449 A1
(43) Pub. Date: Jan. 14, 2010

(54) Cannula Stabilization Device, System, And Method Of Use

(75) Inventors: Carla Leibowitz, San Carlos, CA (US); Christopher U. Phan, San Leandro, CA (US)

Correspondence Address: MEDTRONIC;Attn: Noreen Johnson - IP Legal Department, 2600 Sofamor Danek Drive, MEMPHIS, TN, 38132, US

(73) Assignee: Kyphon SARL, Neuchatel (CH)

(21) Appl. No.: 12/169,707

(22) Filed: Jul. 9, 2008

(51) Int. Cl. A61M 5/32

(52) U.S. Cl. 604/179

(57) ABSTRACT

A cannula stabilization device, systems and kits comprising the cannula stabilization device, and methods of using the cannula stabilization device can include a collar removably securable about a longitudinal axis of a cannula. The collar can include a locking mechanism adapted to secure the collar about the cannula. When the collar is positioned on the skin, the collar can stabilize the cannula from at least axial movement in a distal direction. Embodiments of the devices, systems, kits, and methods are useful for stabilizing a cannula during minimally invasive surgery or other percutaneous procedures.

### FIELD OF INVENTION

The following description relates to a cannula stabilization device, systems and kits comprising a cannula stabilization device, and methods for using a cannula stabilization device.

### BACKGROUND

Conventional surgical procedures for pathologies and/or trauma located deep within the body can cause significant trauma to intervening tissues. Open surgical procedures often require a long incision, extensive muscle stripping, prolonged retraction of tissues, denervation, and devascularization of tissue in order to access a surgical site. Most of these surgeries require several hours of recovery room time and several weeks of post-operative recovery time due to the use of general anesthesia and the destruction of tissue during the surgical procedure. In some cases, these invasive procedures may lead to permanent scarring and pain.

Minimally invasive alternatives, such as endoscopic techniques, reduce pain, post-operative recovery time, and the destruction of healthy tissue. In minimally invasive surgery, the site of pathology is accessed through portals or a small incision rather than through a significant incision, thus preserving the integrity of intervening tissues. These minimally invasive techniques also often require only local anesthesia. The avoidance of general anesthesia can reduce post-operative recovery time and the risk of complications.

Minimally invasive surgical techniques are particularly desirable for spinal and neurosurgical applications because of the need for access to locations deep within the body and the danger of damage to vital intervening tissues. In contrast, a common open procedure for disc herniation, laminectomy followed by discectomy, requires stripping or dissection of the major muscles of the back to expose the spine. In a posterior approach, tissue including spinal nerves and blood vessels around the dural sac, ligaments, and muscle must be retracted to clear a pathway from the skin to the disc. These procedures normally take at least one to two hours to perform under general anesthesia and require post-operative recovery periods of at least several weeks. In addition to the long recovery time, the destruction of tissue is a major disadvantage of open spinal procedures. As a result, many patients may be reluctant to seek surgery as a solution to pain caused by spinal conditions.

In order to reduce the post-operative recovery time and pain associated with spinal and other procedures, micro-surgical techniques have been developed. For example, in micro-surgical discectomies, the disc can be accessed by creating a pathway from the surface of the patient's back to the disc through a percutaneous puncture or small incision. Small diameter micro-surgical instruments may be passed through the puncture access or small incision and between two vertebrae and into the disc. The intervening tissues are disrupted less because the incision and the exterior-to-interior pathway are smaller. Although these micro-surgical procedures are less invasive, they may still involve some risk of complications, such as inadvertent movement of a surgical instrument into an undesired location in or near a surgical site.

Minimally invasive surgery typically utilizes a tubular structure known as a cannula which is inserted into a puncture access or small incision in the body. The cannula holds the puncture access or incision open and serves as a conduit extending between the exterior of the body and the local area inside the body where the surgery is to be performed. Through the conduit, various surgical devices may be inserted and removed. Stationary placement of a cannula can be critical to the success of the surgery as well as the safety of a patient.

Conventional techniques utilized for stabilizing a cannula at a surgical site include, for example, anchoring systems that utilize a series of arms and fixtures that can attach to the operating table or other stationary structure. The arms and fixtures can extend from the attachment point on the operating table to a position above a patient's body. The arms and/or fixtures can be attached to a cannula to hold the cannula in place relative to the surgical site. The arms can provide an obstruction to the surgeon when performing a procedure. Other techniques to stabilize a cannula at a surgical site include manually grasping the access cannula by either the surgeon or another individual assisting with the procedure.

Thus, there is a need for devices, systems, and methods adapted to stabilize a cannula at a surgical site that reduce the risk of patient trauma and that do not substantially impede tasks and/or procedures performed by an operator. There is a need for devices, systems, and methods that provide for such advantages in percutaneous, minimally invasive surgery useful in a variety of applications and approaches.

### SUMMARY

Described herein are embodiments of a cannula stabilization device, systems and kits comprising a cannula stabilization device, and methods for using a cannula stabilization device. Such embodiments may be useful for stabilizing a cannula while performing minimally invasive surgery or for accessing an internal organ or tissue in a human or animal.

In some embodiments, a cannula stabilization device can comprise a collar having a locking mechanism that can be adapted to secure the collar to a structure in a particular dimension. In some embodiments, the collar can be removably securable about a longitudinal axis of a cannula. The locking mechanism can be adapted to secure the collar about the cannula. The collar can be placed on a patient's skin and secured about the cannula such that the collar stabilizes the cannula from at least moving axially in the distal direction.

In some embodiments, the cannula stabilization device can comprise an anchoring mechanism adapted to stabilize the device on the patient's skin. In some embodiments, the anchoring mechanism can be releasably adhered to the skin. Some embodiments can include an anchoring mechanism comprising a plurality of legs extending downward from the collar in a substantially axial direction. The legs can be adjustable such that a portion of each leg can be extended in a substantially perpendicular direction from the collar. In some embodiments, the legs can be adjustable to conform to a patient's anatomy to provide an anchor. In some embodiments, when the device is positioned and/or stabilized on the patient's skin with the anchoring mechanism, the device can stabilize the cannula from axial movement in a proximal direction and/or distal direction.

Other embodiments can include a system and/or kit comprising a cannula stabilization device as described herein. Such a system and/or kit may further comprise additional components, for example, a surgical access cannula and/or surgical instruments. In some embodiments, a cannula stabilization system can comprise a cannula and a collar removably securable about a longitudinal axis of the cannula. The collar can comprise a locking mechanism adapted to secure the collar about the cannula. When positioned on the patient's skin and secured about the cannula, the collar can stabilize the cannula from at least axial movement in a distal direction.

Additional embodiments described herein include a method for stabilizing a cannula at a surgical site. Some embodiments of such a method can include placing a desired portion of the cannula, such as a distal end of a cannula, at the surgical site; providing a cannula stabilization device, as described herein, comprising a collar having a locking mechanism adapted to removably secure the collar about the cannula; positioning the collar on a patient's skin; and securing the collar to the cannula by activating, or engaging, the locking mechanism. In some embodiments, methods can include anchoring the stabilization device to a patient's skin.

Features of a cannula stabilization device, systems, kits, and/or methods of using a cannula stabilization device may be accomplished singularly, or in combination, in one or more of the embodiments. As will be realized by those of skill in the art, many different embodiments of a cannula stabilization device, systems, kits, and/or methods of using a cannula stabilization device are possible. Additional uses, advantages, and features of the cannula stabilization device, systems, kits, and/or methods of using a cannula stabilization device are set forth in the illustrative embodiments discussed in the detailed description herein and will become more apparent to those skilled in the art upon examination of the following.

### BRIEF DESCRIPTION OF DRAWINGS

FIG. 1 is a perspective view of an illustrative embodiment of a cannula stabilization device showing a collar having a locking mechanism and secured about a cannula.

FIG. 2A is a perspective view of the collar having the locking mechanism shown in FIG. 1 in an unsecured configuration.

FIG. 2B is a perspective view of the collar having the locking mechanism shown in FIGS. 1 and 2A in a secured configuration.

FIG. 3 is a perspective view of an embodiment depicting the cannula stabilization device in FIG. 1 secured about a cannula, having a distal end of the cannula positioned in a surgical site.

FIG. 4 is a perspective view of an embodiment depicting a cannula stabilization device secured to a cannula, the device comprising a collar, a locking mechanism, and an anchoring mechanism.

FIG. 5A is a perspective view of an embodiment depicting the collar, the locking mechanism, and the anchoring mechanism shown in FIG. 4 in an unsecured configuration.

FIG. 5B is a perspective view of the collar, the locking mechanism, and the anchoring mechanism shown in FIGS. 4 and 5A in a secured configuration.

FIG. 6 is a perspective view of an embodiment depicting the cannula stabilization device of FIGS. 4 to 5B secured about a cannula with a distal end of the cannula positioned in a surgical site.

FIG. 7A is a perspective view of an embodiment depicting a cannula stabilization device having a collar and an anchoring mechanism having a plurality of adjustable legs extending in a substantially axial direction from the collar.

FIG. 7B is a perspective view of the device shown in FIG. 7A, having legs of the anchoring mechanism adjusted to extend in a substantially perpendicular direction from the collar.

FIG. 8A is a perspective view of another embodiment of a cannula stabilization device having a collar, a locking mechanism, and an anchoring mechanism, the collar being secured to a cannula.

FIG. 8B is a top elevational view of the embodiment shown in FIG. 8A, depicting the device in an unsecured configuration.

FIG. 8C is a top elevational view of the embodiment shown in FIGS. 8A and 8B, depicting the device in a secured configuration.

FIG. 9 is a perspective view of another embodiment of a cannula stabilization device having a collar, a locking mechanism, and an anchoring mechanism.

FIG. 10A is a perspective view of another embodiment of a cannula stabilization device having a collar, a locking mechanism, and an anchoring mechanism, and being unsecured to a cannula.

FIG. 10B is a top elevational view of the embodiment shown in FIG. 10A, depicting the device in a secured configuration.

FIG. 11A is a perspective view of another embodiment of a cannula stabilization device having a collar, a locking mechanism, an anchoring mechanism, and support arms.

FIG. 11B is a perspective view of the embodiment shown in FIG. 11A, depicting the device in an alternate orientation.

FIG. 11C is a perspective view of the cannula stabilization device in FIGS. 11A and 11B secured about a cannula with a distal end of the cannula positioned in a surgical site.

FIG. 12A is a perspective view of another embodiment of a cannula stabilization device having collar, a locking mechanism, an anchoring mechanism, and a clamp shown in an unsecured configuration.

FIG. 12B is a top elevational view of the embodiment in FIG. 12A, shown positioned about a cannula and depicting the collar in an unsecured configuration.

FIG. 12C is a top elevational view of the embodiment shown in FIGS. 12A and 12B, showing the clamp affixed to the collar and depicting the collar in a secured configuration.

FIG. 13 is a perspective view of another embodiment of a cannula stabilization device comprising a collar having a locking mechanism, and plate having a channel through which a portion of the collar can extend.

FIG. 14A is a perspective view of an illustrative embodiment of a cannula stabilization device depicting the collar having a locking mechanism and a hemispherical skirt, the device shown in an unsecured configuration.

FIG. 14B is a perspective view of an embodiment of a cannula stabilization device depicting the collar shown in FIG. 14A in a secured configuration.

FIG. 15A is a perspective view of the proximal surface of the plate shown in FIG. 13.

FIG. 15A is a perspective view of the distal surface of the plate shown in FIG. 13.

FIG. 16 is a flow chart of an embodiment of a method illustrating steps for using a cannula stabilization device.

FIG. 17 is a perspective view of an illustrative embodiment depicting a cannula stabilization device secured to a cannula, the device comprising a collar and a locking mechanism.

FIG. 18A is a perspective view of the cannula stabilization device shown in FIG. 17, having a collar and a locking mechanism in an unsecured configuration.

FIG. 18B is a perspective view of the collar shown in FIGS. 17 and 18A in a partially-closed, unsecured configuration.

FIG. 18C is a perspective view of the collar shown in FIGS. 17, 18A, and 18B in a secured configuration.

### DETAILED DESCRIPTION

Described herein are embodiments of a cannula stabilization device, systems and kits comprising a cannula stabilization device, and methods of using a cannula stabilization device. Such embodiments can be useful for performing minimally invasive surgery and/or other diagnostic and/or therapeutic procedures.

As used in this specification and the appended claims, the singular forms “a,” “an,” and “the” include plural referents unless the context clearly dictates otherwise. Thus, for example, the term “a projection” is intended to mean a single projection or a combination of projections. As used in this specification and the appended claims, “proximal” is defined as nearer to a point of reference such as an origin, a point of attachment, or the midline of the body. As used in this specification and the appended claims, “distal” is defined as farther from a point of reference, such as an origin, a point of attachment, or the midline of the body. Thus, the words “proximal” and “distal” refer to direction nearer to and farther from, respectively, an operator (for example, surgeon, physician, nurse, technician, etc.) who inserts a medical device into a patient, with the tip-end (i.e., distal end) of the device inserted inside the patient's body. For example, the end of a medical device inserted inside the patient's body is the distal end of the medical device, while the end of the medical device outside the patient's body is the proximal end of the medical device.

In some embodiments, a cannula stabilization device can comprise a collar having a locking mechanism that can be adapted to secure the collar to a structure in a particular dimension. In some embodiments, the collar can be removably securable about a longitudinal axis of a cannula. The locking mechanism can be adapted to secure the collar about the cannula. The collar can be positioned on a patient's skin and secured about the cannula such that the collar stabilizes the cannula from at least moving axially in the distal direction. The collar can have a larger cross sectional dimension than the cannula and any corresponding incision or puncture opening through which the cannula extends. Thus the collar can provide a physical structure that prevents the collar and the attached cannula from axial movement in at least the distal direction.

In some embodiments, the cannula stabilization device can comprise an anchoring mechanism adapted to stabilize the device on the patient's skin. Some embodiments can include an anchoring mechanism comprising a plurality of legs extending downward from the collar in a substantially axial direction. In some embodiments, the legs can be adjustable such that a portion of each leg can be extended in a substantially perpendicular direction from the collar. In some embodiments, the legs can be removably adhered to the patient's skin. In some embodiments, when the device is stabilized on the patient's skin with the anchoring mechanism, the device can stabilize the cannula from axial movement in a proximal direction and/or distal direction.

Some embodiments can include a system and/or kit comprising a cannula stabilization device as described herein. The system and/or kit may further comprise additional components, for example, a surgical access cannula and/or surgical instruments. In some embodiments, a cannula stabilization system can comprise a cannula and a collar removably securable about a longitudinal axis of the cannula. The collar can comprise a locking mechanism adapted to secure the collar about the cannula. When positioned on the patient's skin and secured about, or locked upon, the cannula, the collar can stabilize the cannula from at least axial movement in a distal direction.

Other embodiments can include a method for stabilizing a cannula at a surgical site. Some embodiments of such a method can include placing a desired portion of the cannula, such as a distal end of a cannula, at the surgical site; providing a stabilization device as described herein, comprising a collar having a locking mechanism adapted to removably secure the collar about the cannula; positioning the collar on a patient's skin; and securing the collar to the cannula by activating, or engaging the locking mechanism. In some embodiments, methods can include anchoring the device to a patient's skin.

Placement and positioning of a cannula during a surgical procedure can be critical to the success of the surgery as well as to the safety of a patient. Likewise, movement of a cannula during a surgical procedure can be potentially dangerous. Therefore, it can be important to eliminate and/or minimize axial movement of a cannula, particularly in a distal direction. Conventional cannula stabilization devices and methods aimed at minimizing movement of a cannula can often be expensive, complex, and obstructive. For example, conventional methods and devices may reduce the efficiency of an operator or impede an operator's movements.

Described herein are embodiments that can provide customizable and unobtrusive devices to stabilize a cannula. Some embodiments described herein can provide a device which protects delicate anatomy, such as nerve tissue, from accidental trauma that may be caused by axial movement of a cannula. Some embodiments can help ensure proper, desired cannula placement during a surgical procedure and/or other diagnostic and/or therapeutic procedure. Additionally, some embodiments can provide a device that does not obstruct or impede an operator's movement, for example, with arms and/or fixtures mounted to an operating or procedure table. Some embodiments may allow a surgeon's hand to be free from holding the cannula. Also, embodiments described herein may provide a device that can be efficiently sterilized to reduce procedure preparation times. In some embodiments, the device may be disposable.

In some embodiments, the cannula stabilization device can comprise a collar that can be removably secured about a cannula along the longitudinal axis of the cannula. The longitudinal axis of the cannula extends from a proximal end to the distal end of the cannula, along the length of the cannula. The collar can be positioned around the outer circumference or perimeter of the cannula. The collar can have an adjustable cross-sectional dimension. In some embodiments, the collar can comprise a generally circular cross-sectional dimension. In other embodiments, the collar can comprise a generally triangular cross-sectional dimension. The collar can comprise an inner surface and an outer surface. When positioned about a cannula, the inner surface of the collar can contact an outer surface of the cannula. In some embodiments, substantially the entire inner surface of the collar can contact the outer surface of the cannula. In some embodiments, the collar can be adjusted to have an inner cross-sectional dimension that is substantially equal to a outer cross-sectional dimension of the cannula. In other embodiments, sections of the inner surface of the collar less than the entire inner surface can contact the outer surface of the cannula.

In some embodiments, the collar can comprise a locking mechanism. The locking mechanism can be adapted to secure the collar about the cannula. In some embodiments, the locking mechanism can secure the collar to an inner cross-sectional dimension substantially equal to the outer cross-sectional dimension of the cannula.

In some embodiments, the locking mechanism can comprise an engaging member and a mateably engaging surface. The engaging member can be interlocked into the engaging surface creating a releasably secure fit, or engagement between the member and surface. In some embodiments, the engaging member can further comprise an arm having a protrusion extending substantially perpendicular from the arm. The engaging surface can comprise a cavity. In some embodiments, the protrusion can be interlocked with the cavity to create a releasably secure fit or lock. In some embodiments, the engaging member can snap into the engaging surface such that the engaged member and engaging surface are removably secured to each other.

In other embodiments, the locking mechanism can comprise a screw and nut. In some embodiments, the screw can extend through a mated opening defined by two flanges of a collar. Each flange can extend outwardly from each of two edges of the collar. The flanges can comprise mateable openings. With the screw extended through the mated openings, the nut can be tightened upon the screw to secure the collar about the cannula. By tightening the screw, the distance between the flanges can be decreased, thus also decreasing the inner dimensions of the collar such that the inner surface of the collar securably contacts the outer surface of the cannula. The screw and nut can be tightened to a position such that collar can be secured about the cannula thus preventing any axial movement of the cannula with respect to the collar.

In yet other embodiments, the locking mechanism can comprise a clamp positionable over flanges of a collar. The collar can comprise a flange extending outwardly from each of two edges of the collar. In some embodiments, the clamp can be configured to be slidingly positionable upon the flanges. In some embodiments, the flanges can have an original, unlocked configuration having a defined distance between the inner surface of the flanges. When the flanges are in the original configuration, the collar can have internal dimensions that do not sufficiently contact the cannula to secure the collar about the cannula; thus in the original, unlocked configuration the cannula can freely move in an axial direction. To secure the collar about the cannula, the position of the flanges can be adjusted such that the inner surface of the flanges approach and/or contact each other, and a clamp can be positioned upon the flanges so as to compress the inner surface of the collar onto the outer surface of the cannula.

In some embodiments, the clamp can be adjustable. In other embodiments, the clamp can be non-adjustable. The clamp can comprise a sufficiently rigid material that withstands forces of the flanges to return to their original configuration. In some embodiments, the clamp can comprise a C-shaped clamp or U-shaped clamp.

In one embodiment, the locking mechanism can comprise an inherent spring-bias in the collar itself. For example, the collar can exhibit a natural (i.e., at-rest) oval aperture, such that the short dimension of the oval aperture is less than the diameter of the cannula. As a result, when placed on the cannula (e.g., by first squeezing the collar across the long dimension of the oval to widen the short dimension), the collar naturally grips the cannula to provide retention/positioning functionality. Various other spring-loaded clamping configurations will be readily apparent.

In some embodiments, when the collar is positioned on a patient's skin, the collar can stabilize the cannula from at least axial movement in a distal direction. The cannula can be inserted to a surgical site and adjusted to a desired position relative to the surgical site. In some embodiments, the collar, in its unlocked configuration, can be positioned on the patient's skin and about the cannula along its longitudinal axis. When the collar is in a desired position along the cannula, the locking mechanism can be engaged to secure the collar about the cannula. In some embodiments, the collar can comprise an outer dimension that can be greater than the dimensions of an access incision or puncture through which the cannula extends, thus preventing axial movement in the distal direction.

With the collar positioned on a patient's skin and secured about the cannula, an operator can have a greater level of confidence in the placement of the distal portion, for example, distal end of the cannula, relative to the surgical site. Axial movement of the cannula in the distal direction can be prevented by the physical obstruction that the collar can provide. Axial movement of the cannula in the proximal direction can be detected by observing any distance between the collar and the skin. When the collar is secured about the longitudinal axis of the cannula, the relative positions of the collar and the cannula remain constant. Thus, any axial movement of the cannula in the proximal direction results in axial movement of the collar in a proximal direction. In some embodiments, the proximal axial movement of the collar can be detected as the distance between the collar and skin increases. In contrast, detection of axial movement in the proximal direction by a cannula, not having a collar secured, may be difficult without the visual frame of reference provided by the collar.

In some embodiments, the cannula stabilization device can comprise an anchoring mechanism. The anchoring mechanism can be adapted to stabilize the device on the patient's skin. In some embodiments, the anchoring mechanism can be integral with or attached to the collar.

In some embodiments, the anchoring mechanism can be releasably attached to the patient's skin. When attached, or adhered, to the patient's skin, the anchoring mechanism can stabilize the cannula from axial movement in a proximal direction. For example, tape may be applied to the patient's skin and extended over the anchoring mechanism to retain the anchoring mechanism in contact with the skin. In other embodiments, a releasable adhesive layer may be applied to a distal surface of an anchoring mechanism such that the distal surface of the anchoring mechanism can be secured to the skin upon contact.

In some embodiments, the anchoring mechanism can comprise a plurality of legs extending downward from the collar in a substantially axial direction. Each of the legs can be independently adjustable so that at least a portion of the leg can extend in a substantially perpendicular direction from the collar. In some embodiments, the height of each leg can be adjusted. In some embodiments, the angle in which each leg extends from the collar can be adjusted. For example, the collar can have three legs, each of which can be adjusted or bent to a different length and/or to have a different angle in order to accommodate the irregularities of a patient's body to which the anchoring mechanism can be attached. In some embodiments, the legs can be releasable adhered to a patient's skin. For example, tape may be applied over a proximal surface of each leg extending substantially perpendicular to the collar. In some embodiments, a distal surface of each leg extending substantially perpendicular to the collar can comprise a releasable adhesive to temporarily secure the leg to the patient's skin.

In some embodiments, the anchoring mechanism can comprise a skirt. In some embodiments, the skirt can be integrally formed with the collar. In some embodiments, the skirt can be connected to and extend from the collar at the distal region of the collar. In other embodiments, the skirt can be connected to the collar through a support member such as an arm or bracket member. In some embodiments, the skirt can extend at an angle from the collar in an outward direction. For example, the skirt may extend outwardly from the collar in a substantially perpendicular direction.

In some embodiments, the skirt can comprise a ring shaped structure. In other embodiments, the skirt can comprise a hemispherical shaped structure. In yet other embodiments, the skirt can comprise a frustoconical shaped structure.

In some embodiments, the skirt can be releasably adhered to a patient's skin. For example, tape may be applied to the skin and extended over a proximal surface of the skirt so that the skirt can be adhered to the skin. In some embodiments, a distal surface of the skirt can comprise a releasable adhesive layer to temporarily secure the skirt to the patient's skin.

In other embodiments, the anchoring mechanism can comprise a collar having a locking mechanism that can be connected to the anchoring mechanism by a support member. In some embodiments, the support member can comprise a plurality of arms that extend from a ring to the collar. In some embodiments, the support members can be sufficiently rigid to prevent any movement of the collar. In other embodiments, the support members can be attached to the collar such that the collar can be rotated to adjust the orientation of the collar.

In yet other embodiments, the anchoring mechanism can comprise a plate mountable on a patient's skin. The plate can comprise a channel through which at least a portion of the collar can extend. In some embodiments, the channel can be defined by a semi-cylindrical shaped cavity. In such embodiments, the collar can include a distal portion comprising a skirt extending downward and outward from the collar. In some embodiments, the distal portion of the collar can comprise a hemispherical shape. The distal portion of the collar can be adapted to slidingly engage the semi-cylindrical shaped cavity defining the channel such the collar can be moved in a plane along the channel. In embodiments in which the collar is secured to a cannula, the cannula can be moved along a plane of the channel. For example, in particular embodiments, the axial positioning of the cannula can be stabilized by the collar on the patient's skin while the cannula can be moved along the channel in the plate such that the distal portion of the cannula can move along the plane or, or “wag” within, an intervertebral disc. In alternate embodiments, the distal portion of the cannula can move along the plane, or wag within, an intravertebral body, for example as shown in FIG. 3.

In such embodiments, the collar and/or anchoring mechanism can restrict the axial movement of the cannula. The collar can be secured about the cannula to provide a physical structure that may prevent the cannula from being shifted in a distal direction along the longitudinal axis of the cannula. In embodiments having the plate adhered to the skin, the plate can stabilize the cannula from been adjusted in a proximal direction.

The collar and anchoring mechanism can comprise material(s) that provide a balance of rigidity and flexibility to facilitate securing of the cannula stabilization device about the cannula as well as to stabilization of the cannula during use. In addition, materials comprising the cannula stabilization device can facilitate delivery and manipulation of additional surgical tools that may be utilized in connection with a minimal invasive surgical procedure, for example delivery cannulas and expandable members inserted through an access cannula. Such materials can include, for example, vinyl, nylon, polyethylenes, ionomer, polyurethane, and polyethylene tetraphthalate (PET), stainless steel, Kevlar™ material, PEBAX™ material, nickel-titanium alloys (Nitinol™ material), and other metal alloys.

Additional embodiments can include methods for using a cannula stabilization device. In some embodiments, a cannula can be percutaneously inserted into a patient toward a surgical site. When a desired portion, for example, a distal end of the cannula, is properly positioned, a cannula stabilization device can be positioned about the longitudinal axis of the cannula. The collar can comprise a locking mechanism adapted to secure the collar to the cannula. The collar can be positioned about the cannula upon a patient's skin in an unsecured configuration. When positioned on the skin, the locking mechanism can be engaged to secure the collar around the cannula. When the collar is secured around the cannula and in contact with the patient's skin, the cannula can be prevented from axial movement in the distal direction. In some embodiments, the cannula stabilization device may be anchored to the patient's skin. In some embodiments, the device can be anchored utilizing an anchoring mechanism as described herein. In some embodiments, the anchoring can include applying tape extending over the proximal surface of the anchoring mechanism.

Referring now to the figures, label 10 and 10′ refer to the collar. When the collar is labeled as 10′, the collar is shown in its unlocked or unsecured configuration. When the collar is labeled as 10, the collar is shown in the locked or secured configuration.

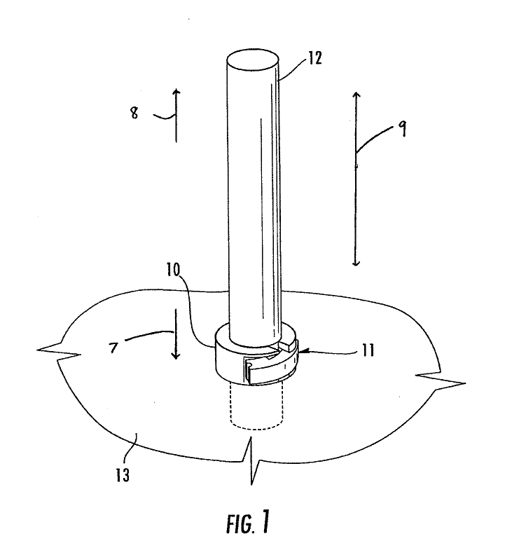

In the embodiments shown in FIGS. 1-3, a stabilization device comprises a collar 10 and a locking mechanism 11. In the embodiment depicted in FIG. 1, the collar 10 can be positioned about the longitudinal axis 9 of a cannula 12. The collar 10 and the cannula 12 can be substantially coaxial. In FIG. 1, the collar 10 can be secured around the cannula 12 and positioned in proximity to a patient's skin 13. The cross-sectional dimension of the collar 10 can be greater than the cross-sectional dimension of the cannula 12. Additionally, the cross-sectional dimension of the collar 10 can be greater than the dimensions of an incision or puncture access (not shown) of the skin 13 through which the cannula 12 extends. Thus, the cannula 12, having the collar 10 secured to it, can be prevented from axial movement in a distal direction 7.

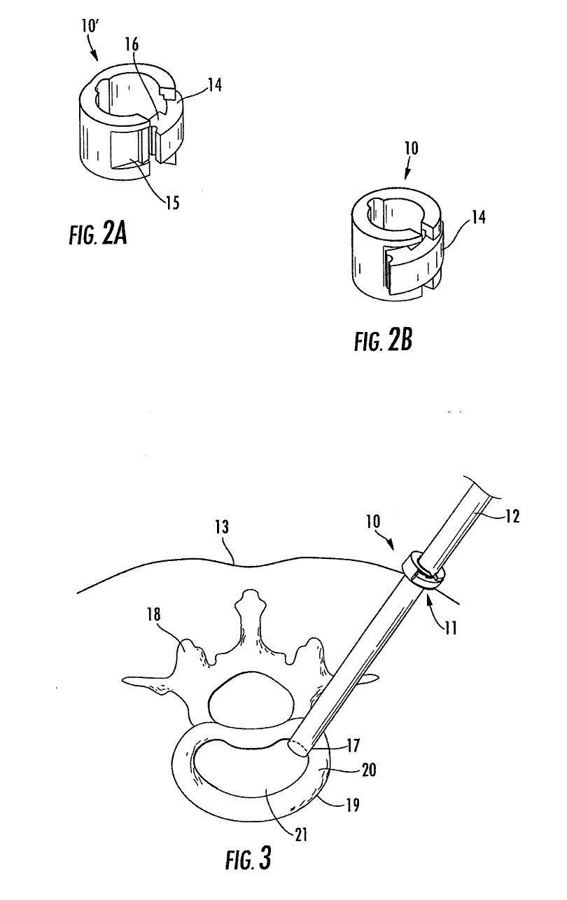

FIG. 2A depicts the collar 10′ in an unsecured or unlocked position. The collar 10′ comprises an arm 14, a protrusion 16, and a cavity 15. The protrusion 16 can be interlocked into the cavity 15 such that a secure connection can be created between the protrusion 16 and the cavity 15. FIG. 2B depicts the collar 10 in the secured or locked configuration with the protrusion 16 interlocked into the cavity 15. In some embodiments, the cannula 12 can be percutaneously positioned. The collar 10′ can be positioned about the longitudinal axis 9 of the cannula 12. In some embodiments, the collar 10′ can be positioned on the patient's skin 13. Once the collar 10′ is positioned on the skin 13, the arm 14 can be adjusted so that the protrusion 16 aligns with the cavity 15. Pressure can be applied to protrusion 16 to snap the protrusion 16 into the cavity 15 resulting in the collar 10 being secured, or locked, about the cannula 12.

FIG. 3 depicts an illustrative embodiment in which the collar 10 has been secured to the cannula 12 positioned at a surgical site. In the embodiment shown in FIG. 3, the surgical site comprises a vertebra 18, including an intravertebral body 19 comprising a cortical bone 20 and cancellous bone 21. The distal end 17 of the cannula 12 can be positioned, for example, in the cancellous bone 21. The collar 10 has been secured to the cannula 12 at the skin 13. The collar 10 prevents the distal end 17 of the cannula 12 from axially moving more distally in the cancellous bone 21.

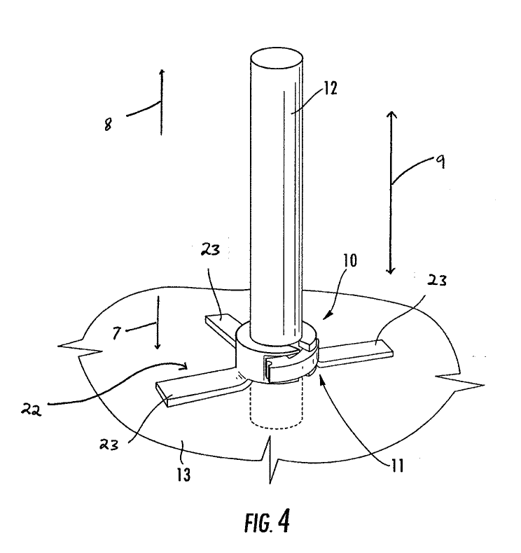

FIGS. 4 to 6 depict an additional embodiment of a cannula stabilization device similar to that shown in FIGS. 1-3. In FIG. 4, the cannula stabilization device comprises the collar 10 and an anchoring mechanism 22 comprising a plurality of legs 23. The collar 10 can be secured about the longitudinal axis 9 of the cannula 12. In the embodiment shown, the collar 10 can be positioned on the patient's skin 13. As shown in FIGS. 4-6, the legs 23 of anchoring mechanism 22 can extend substantially perpendicular to collar 10. In some embodiments, the legs 23 can be releasably adhered to the skin 13. For example, tape (not shown) can be applied to extend over the proximal surface of leg such that the leg can be secured to the skin 13 with the tape. In some embodiments, a releasably adhesive layer can be applied to the distal surface of each leg such that the distal surface of the legs can be secured to the skin 13.

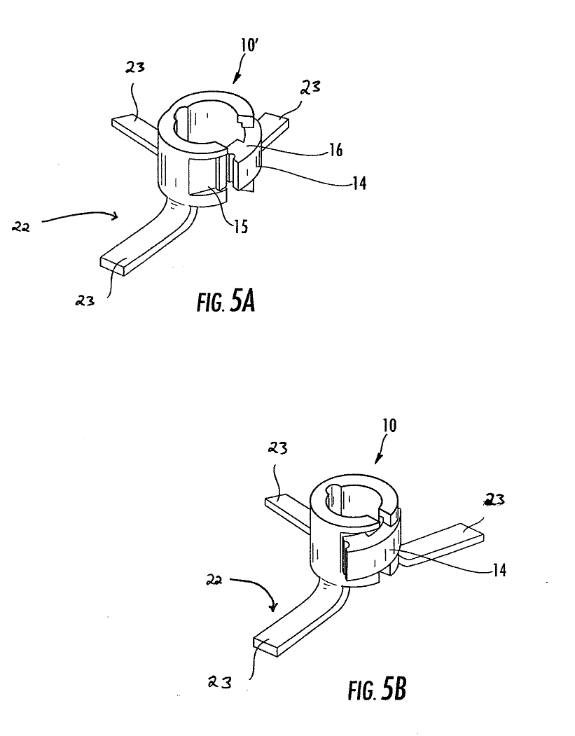

FIG. 5A depicts the collar 10′ in its original, unsecured or unlocked position. The collar 10′ comprises the arm 14, the protrusion 16, the cavity 15, and the anchoring mechanism 22 comprising a plurality of legs 23. The protrusion 16 can be interlocked into the cavity 15 such that a secure connection can be created. FIG. 5B depicts collar 10 in the secured or locked configuration with the protrusion 16 interlocked into the cavity 15. In some embodiments, once a cannula 12 is percutaneously positioned, the collar 10′ can be positioned about the longitudinal axis 9 of the cannula 12. In some embodiments, the collar 10′ can be positioned on a patient's skin 13. When positioned on the patient's skin 13, the legs 23 of the anchoring mechanism 22 can be in contact with the skin 13. Once the collar 10′ is positioned on the skin 13, the arm 14 can be adjusted so that the protrusion 16 aligns with the cavity 15. Pressure can be applied to the protrusion 16 to snap the protrusion 16 into the cavity 15 resulting in the collar 10 being secured, or locked.

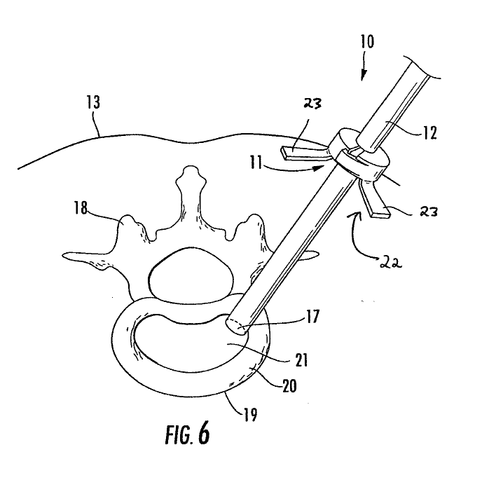

FIG. 6 depicts an illustrative embodiment where the collar 10 comprising the anchoring mechanism 22 having the plurality of legs 23 has been secured to a cannula 12 positioned at a surgical site. As shown in the embodiment in FIG. 6, the surgical site can comprise a vertebra 18, including an intravertebral body 19 comprising a cortical bone 20 and cancellous bone 21. The distal end 17 of cannula 12 can be positioned, for example, in the cancellous bone 21. The collar 10 can be secured to cannula 12 at skin 13. The collar 10 prevents the distal end 17 of the cannula 12 from axially moving more distally in the cancellous bone 21. In some embodiments, the anchoring mechanism 22 comprising legs 23 can be releasable secured to the skin 13. For example, tape may be applied to the skin 13 and extend over the proximal surface of the legs, thus securing the legs to the skin 13. The anchoring mechanism 22 can stabilize the distal end 17 of the cannula 12 from axial movement in the proximal direction and/or distal direction within the cancellous bone 21 or partially out of the cancellous bone 21.

FIGS. 7A and 7B depict a cannula stabilization device comprising a collar 10 and anchoring mechanism 22 having a plurality of legs 23. The collar 10 comprises a locking mechanism 11. Legs 23 can be adjusted or bent such that a portion of the legs 23 extend substantially perpendicular to the longitudinal axis of the collar 10 as shown in FIG. 7B. In some embodiments, each of the legs 23 can be adjusted independently. In some embodiments, legs 23 can be oriented at a range of angles with respect to the longitudinal axis of the collar. For example, legs 23 can be oriented substantially perpendicular to the longitudinal axis of collar 10. Other embodiments can comprise the legs 23 being oriented at an acute or obtuse angle with respect to the longitudinal axis of collar 10. By modifying the height in which the legs 23 are bent, the height in which the collar 10 is positioned can be adjusted. The independent adjustment of the legs 23 provides a mechanism for the surgeon to account for irregularities in anatomical regions of the patient and different anatomical shapes of different patients.

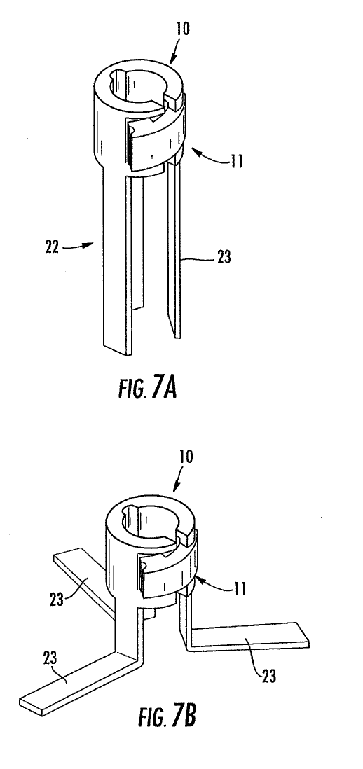

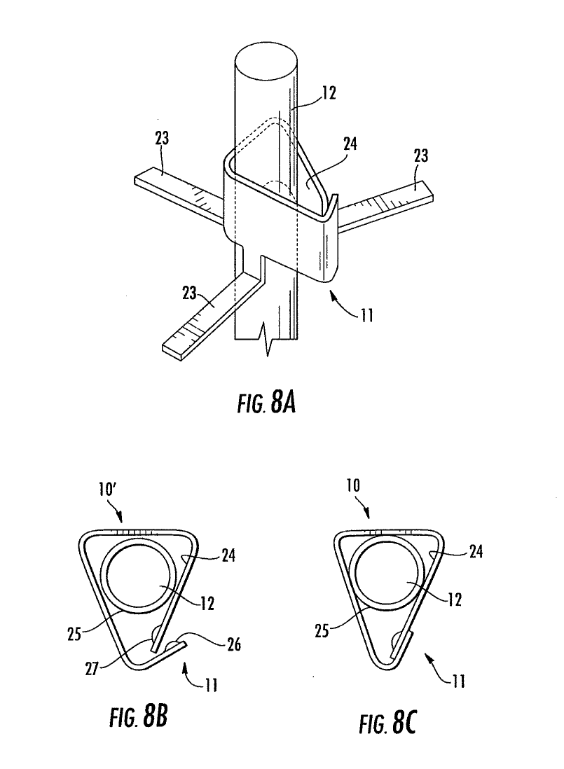

FIGS. 8A to 8C depict a cannula stabilization device comprising a collar 10 positioned about a longitudinal axis of a cannula 12. The collar 10 has a generally triangular-shaped cross-section. The device shown in FIG. 8A further depicts an anchoring mechanism comprising a plurality of legs 23. The collar can comprise an inner surface 24 which can contact the cannula 12 in a plurality of positions.

FIG. 8B depicts a top elevational view of the collar 10′ about the cannula 11. FIG. 8B depicts an inner surface 24 of collar 10′ (in the unsecured configuration) in proximity to an outer surface 25 of cannula 12. In the unsecured configuration, the inner surface 24 of the collar 10′ does not contact the outer surface 25 of the cannula in a sufficient manner, for example, in a sufficient number of contact points to secure the collar 10′ about the cannula 12. While the collar 10′ is in the unsecured configuration about the cannula 12, the cannula 12 can be adjusted freely in the axial direction within the collar 10′. The locking mechanism 11 can comprise a protrusion 26 and a cavity 27. The protrusion 26 and the cavity 27 can be interlockable so as to lock the collar 10 in the secured configuration.

FIG. 8C depicts a top elevational view of collar 10 secured about the cannula 12. The protrusion 26 can be interlocked with the cavity 27 thus locking the locking mechanism 11. As the locking mechanism 11 is locked, the locking mechanism 11 secures the collar 10 about the longitudinal axis of the cannula 12. The inner surface 24 of the collar 10 contacts the outer surface 25 of the cannula 12 such that the cannula 12 can no longer move axially relative to the collar 10. In some embodiments, when positioned on a patient's skin 13, the collar 10 can prevent the axial movement of cannula 12 in the distal direction 7. In some embodiments, when positioned on a patient's skin 13, the anchoring mechanism 22 can be releasably adhered to the skin so as to prevent the axial movement of cannula 12 in the proximal direction 8 and/or distal direction 7.

FIG. 9 depicts another embodiment of a cannula stabilization device. The cannula stabilization device comprises a collar 10 and a locking mechanism 11. The locking mechanism 11 can operate similar to the embodiments described in FIGS. 1-7. The embodiment of the cannula stabilization device shown in FIG. 9 includes an anchoring mechanism comprising a skirt 28. In FIG. 9, the skirt 28 comprises a flange. In some embodiments, the skirt 28 can be releasably adhered to a patient's skin 13. For example, tape can be applied to the patient's skin 13 and extended over a portion of the skirt 28 to secure the device to the patient's skin 13.

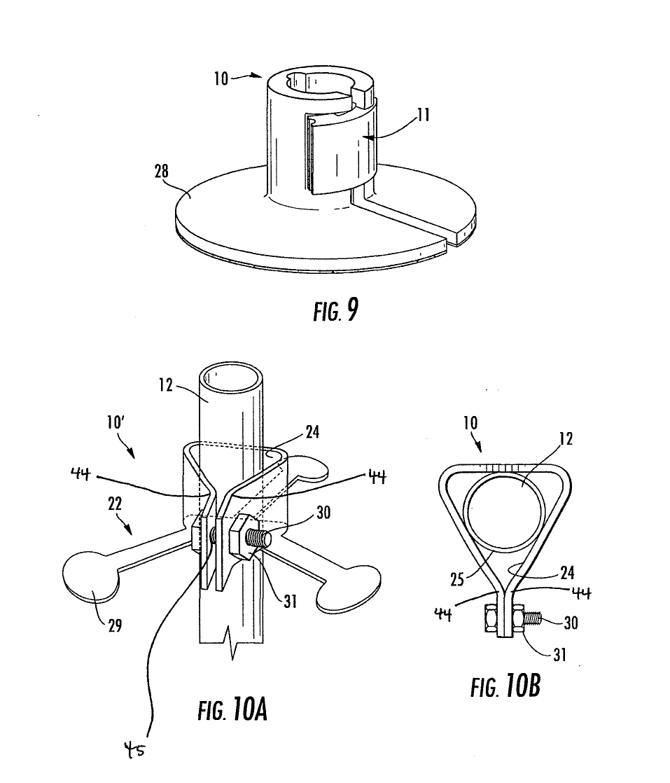

FIGS. 10A and 10B depict an embodiment of a cannula stabilization device including a locking mechanism comprising a screw 30 and nut 31. In some embodiments, the collar 10 comprises a flange extending outwardly from each of two edges 44 of the collar 10. Each flange has a mateable opening 45 through which the screw 30 can extend. The nut 31 can be threaded onto the screw 30. When the nut 31 is not tightened upon the screw 30 as in the collar 10′, the cannula 12 can freely move in an axial direction in relation to the collar 10′. As shown in FIG. 10B, by tightening the nut 31 upon the screw 30, the collar 10 can be drawn tightly about the cannula 12 so as to be secured about the cannula 12.

Upon tightening the nut 31 onto the screw 30, the distance between the flanges can be decreased. As the flanges are adjusted to a position in proximity to each other, the inner surface 24 of the collar 10 contacts the outer surface 25 of cannula 12.

The embodiment in FIG. 10A further comprises an anchoring mechanism 22 comprising a plurality of legs having feet 29. The feet 29 can provide a greater surface area to stabilize the device upon a patient's skin 13. In some embodiments, the feet 29 can be releasably adhered to the skin 13. For example, tape can be applied to the skin 13 and extend over the feet 29 so as to adhere the device to the skin 13. The feet 29, in conjunction with the collar 10′ and legs 23, can prevent axial movement of the cannula in a proximal direction 8 and/or distal direction 7.

In yet another embodiment, FIGS. 11A and 11B depict a cannula stabilization device comprising a collar 10 and an anchoring mechanism 22 comprising a ring 32 and support members 33. The support members 33 can connect the ring 32 and the collar 10. In some embodiments, the support members 33 can extend substantially vertically from the ring 32. The support members 33 can be attached to the ring 32 at a point 35 in the ring 32. The support members 33 can be attached to the collar 10 at a point 34. In some embodiments, the collar 10 may be adjusted around an axis between the points 34. As seen in FIG. 11C, the rotation of the collar around the points 34 can adjust the orientation of the cannula 12 extending into a surgical site. The ring 32 can stabilize the device and the cannula 12 from axial movement in the proximal direction 8 and/or distal direction 7. In some embodiments, the orientation of collar 10 can be limited by the dimensions of the ring 32 (i.e., by the inner diameter of the ring).

FIG. 11C depicts an illustrative embodiment in which the collar 10 comprises an anchoring mechanism having the ring 32 and support members 33 that can be secured to a cannula 12 positioned at a surgical site. In the embodiment shown in FIG. 11C, the surgical site comprises a vertebra 18, including an intravertebral body 19 comprising a cortical bone 20 and cancellous bone 21. The distal end 17 of cannula 12 can be positioned, for example, in the cancellous bone 21. As shown in FIG. 11C, the collar 10 can be secured to cannula 12 at the skin 13. In some embodiments, the ring 32 can be releasably adhered to skin 13. The collar 10 and the ring 32 can stabilize the distal end 17 of cannula 12 from axial movement in the distal direction 7 in the cancellous bone 21. The ring 32 and support members 33 attached to the collar 10 can stabilize the distal end 17 of the cannula 12 from axial movement in the proximal direction 8 within the cancellous bone 21 or partially out of the cancellous bone 21. In some embodiments, points 34, 35 can provide a mechanism to rotate and adjust the orientation of the collar 10. In some embodiments, the collar 10 can be locked into a particular orientation about points 34. In other embodiments, collar 10 can have a limited range of angles to orient the cannula 12.

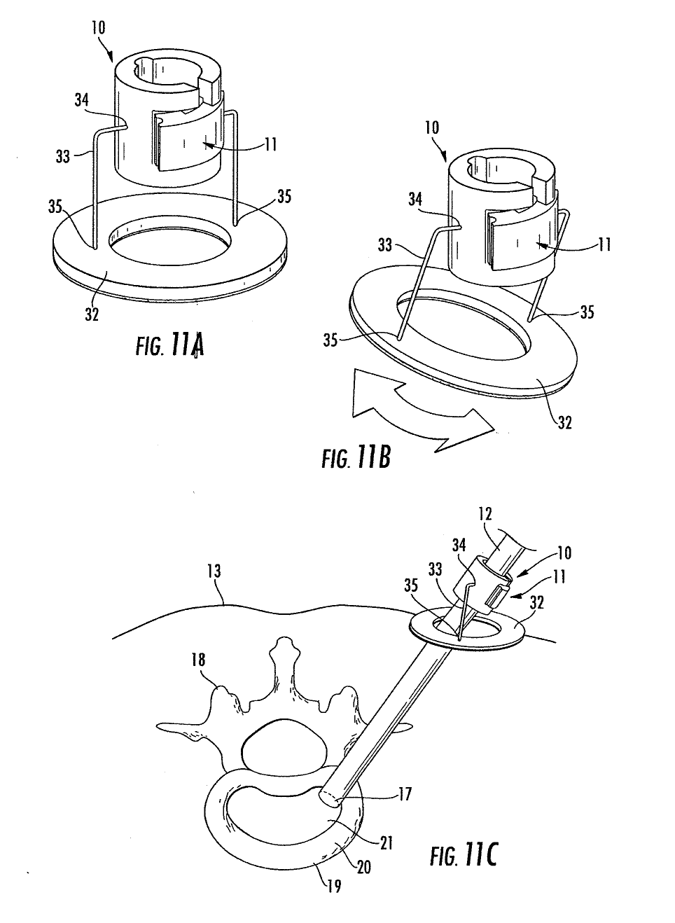

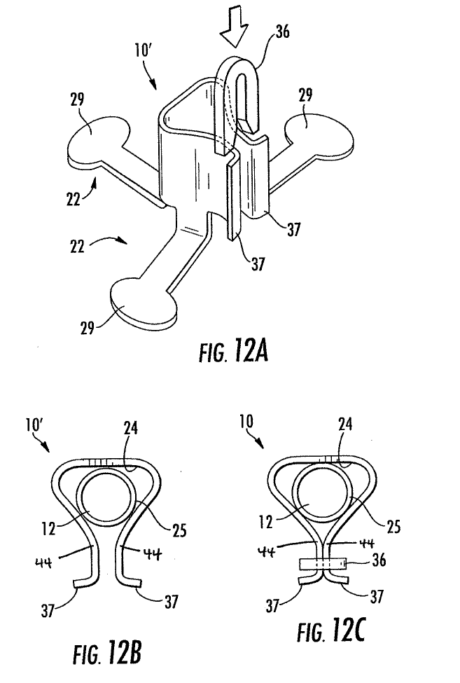

FIGS. 12A to 12C depict another embodiment of a cannula stabilization device comprising a collar 10 having a locking mechanism 11 and an anchoring mechanism 22 comprising feet 29. The locking mechanism can comprise a U-shaped member 36 configured to be slidingly positionable upon flanges 37 extending outward from the collar 10. The flanges 37 can extend outwardly from each of two edges 44 of the collar 10. As shown in FIG. 12B, the flanges 37 can have an unsecured, or open, configuration of having a space between the flanges 37. In such orientation, the inner surface 24 of the collar 10′ does not contact the outer surface 25 of the cannula 12 with sufficient force to secure the relative axial positions of the collar 10′ and cannula 12. In the open, unsecured configuration, the cannula 12 may move freely in an axially direction in the embodiment shown in FIG. 12B.

In the embodiment shown in FIG. 12C, the collar 10 is secured about the cannula 12 such the axial movement of the cannula 12 relative to the collar 10 is prevented. In some embodiments, the flanges 37 can be adjusted with the application of manual pressure so that the inner surfaces of the flanges 37 approach each other. When the flanges 37 are pressed closer to each other, the inner surface 24 of the collar 10 contacts the outer surface 25 of the cannula 12 such that the cannula 12 is stabilized from moving axially relative to the collar 10. The U-shaped member 36 can be slidingly positioned upon the flanges 37 to secure the collar 10 about the cannula 12. The U-shaped member 36 can comprise a sufficiently rigid material to withstand the forces of the flanges to return to their unsecured, open configuration.

FIGS. 13 to 15B depict another embodiment of a cannula stabilization device. The device depicted in FIG. 13 comprises a collar 10 having a locking mechanism 11 and an anchoring mechanism 22 comprising a plate 37. The plate 37 can be mountable on a patient's skin 13. The plate 37 can comprise a channel 40 through which at least a portion of the collar 10 can extend. The plate 37 can comprise a semi-cylindrical region 41 that defines channel 40.

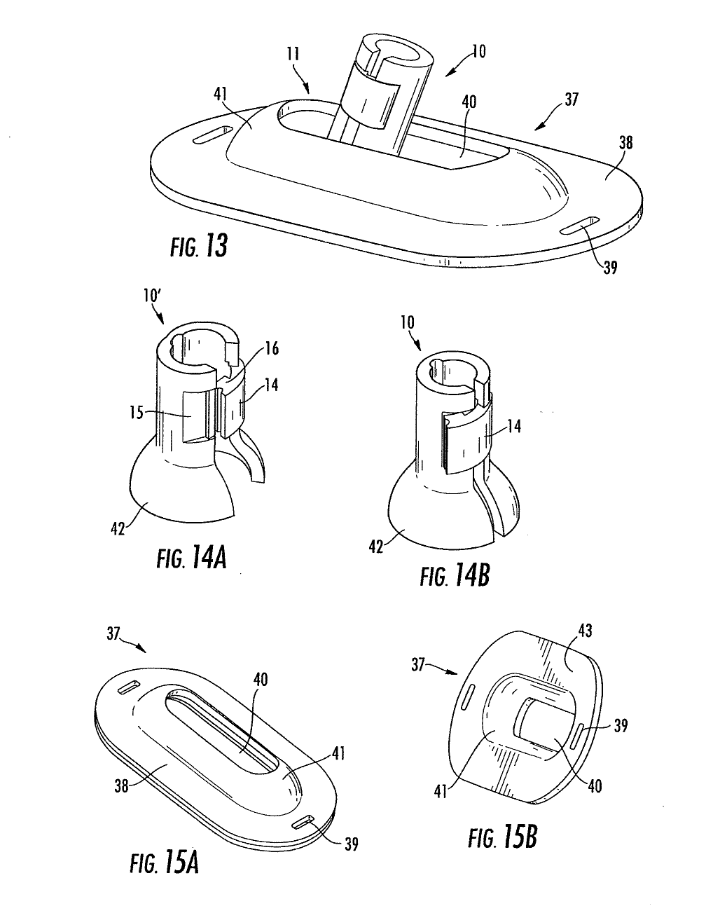

FIG. 14A depicts a collar 10′ in the unlocked configuration comprising a locking mechanism 11 having an arm 14, a cavity 15, and a protrusion 16. When the collar 10′ is positioned about a cannula 12, the protrusion 16 can interlock with cavity 15 such the collar 10 can be secured about the cannula 12. Similar to embodiments described herein, when the collar 10 is secured about the cannula 12, the cannula 12 can be prevented from axial movement in at least the distal direction 7. FIG. 14B depicts the collar 10 in a secured configuration. The collar 10 of FIGS. 14A and 14B further comprises a skirt 42 that extends downward and outward from the collar 10. In some embodiments, the skirt 42 comprises a generally hemispherical shape. In some embodiments, the skirt 42 comprises a complimentary shape to a shape of the semi-cylindrical region section 41 that defines channel 40.

FIGS. 15A and 15B show a perspective view of plate 37 without collar 10 extending through the channel 40. FIG. 15A depicts the proximal surface 38 of plate 37. Opening 39 can provide an opening through which a tying device may be extended. In some embodiments, the tying device can comprise a strap that can be extended around the patient's body to secure the plate 37 in a desired position. FIG. 15B depicts the distal surface 43 of the plate 37. The distal surface of the semi-cylindrical region 41 defines the channel 40.

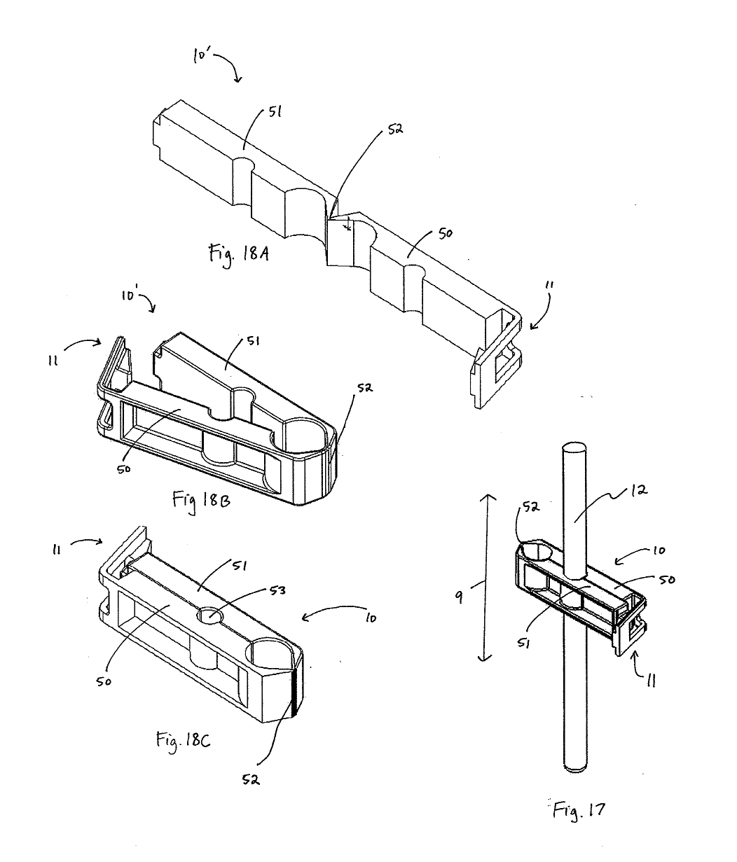

FIGS. 17 to 18C depict an additional embodiment of a cannula stabilization device. In FIG. 17, the cannula stabilization device comprises a collar 10 positioned about longitudinal axis 9 of a cannula 12. The collar comprises a locking mechanism 11, a first arm 50, a second arm 51, and a hinge 52. In some embodiments, the locking mechanism 11 can comprise a protrusion that can be interlocked into a cavity to create a secure connection of the collar 10 around the cannula 12.

FIG. 18A depicts the collar 10′ in an unsecured or unlocked position. The collar 10′ comprises the first arm 50, the second arm 51, the hinge 52, and the locking mechanism 11. In some embodiments the first arm 50 and the second arm 51 can be attached to the hinge 52. In some embodiments, the hinge 52 may be a living hinge. In other embodiments, the hinge 52 may be a discrete component. The first arm 50 and the second arm 51 can rotate around the hinge 52 such that the side walls of the first arm 50 and the second arm 51 can be positioned adjacent to each other. FIG. 18B depicts the collar 10′ in a partially-closed, unsecured configuration where the first arm 50 and/or the second arm 51 have rotated about the hinge 52. FIG. 18C depicts the collar 10 in a secured configuration. The side walls of the first arm 50 and second arm 51 are positioned adjacent to each other. In the embodiment shown in FIG. 18C, the first arm 50 and the second arm 51 of collar 10 define an opening 53. The locking mechanism 11 can be secured such that the first arm 50 and the second arm 51 remain adjacent to each other defining the opening 53. When the collar 10 is positioned about the cannula 12, the cannula 12 can extend through the opening 53.

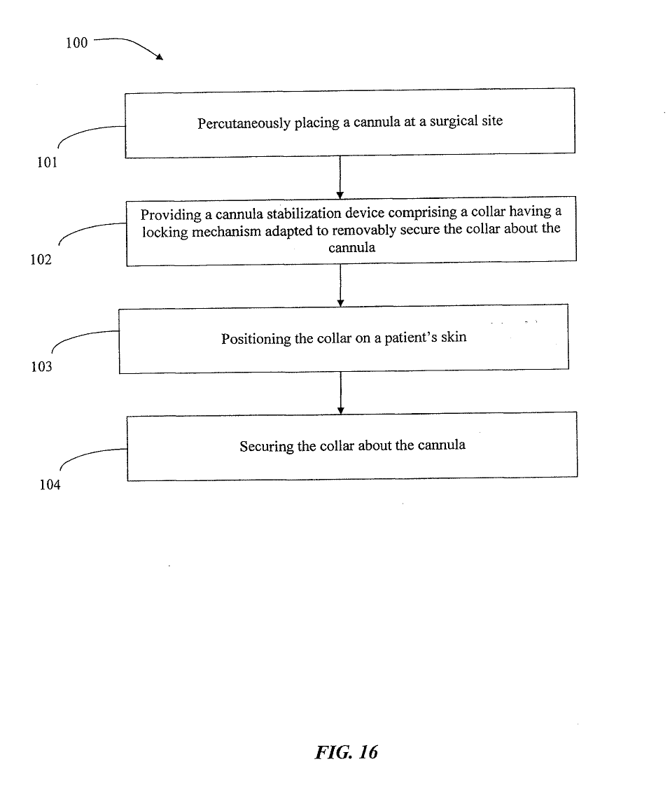

Embodiments can include methods for using a device to stabilize a cannula 12 during a surgical procedure or other treatment of a human or animal. FIG. 16 illustrates an embodiment of such a method 100. In such an embodiment, a cannula 12 may be percutaneously placed at a surgical site (101). With the distal portion of the cannula 12 positioned at the desired site, a stabilization device comprising a collar 10 and locking mechanism 11 can be provided (102). The collar 10 can be positioned about the cannula 12 and positioned upon a patient's skin 13 (103). With the collar 10 positioned upon the patient's skin 13, the collar 10 can be secured about the cannula (104) as to stabilize the cannula from axial movement in at least the distal direction 7. The methods may further comprise securing an anchoring mechanism 22 upon the patient's skin 13. Upon securing the anchoring mechanism 22, the cannula 12 can be stabilized from axial movement in the distal direction 7 and/or proximal direction 8.

Embodiments of a cannula stabilization device, system, kit, and method of using cannula stabilization device can be utilized for facilitating minimally invasive surgical procedures and/or accessing other internal organs or tissues. Some embodiments can be advantageously used in accessing and stabilizing an intravertebral body and/or in accessing, stabilizing, and fusing of a joint, particularly an intervertebral joint. Some embodiments may be applicable for use with various types of joints (for example, intervertebral, ankle, interdigital, etc.) and in various anatomical regions (for example, spine, arms, legs, etc.) of a human or animal body. In the spinal column, the devices and methods disclosed may be used at all intervertebral joints, including those in the cervical, thoracic, and lumbar region.

Although this description refers to particular embodiments, it should be recognized that these embodiments are merely illustrative of novel and non-obvious principles. Those of ordinary skill in the art will appreciate that a cannula stabilization device, system, kit, and methods for using a cannula stabilization device may be constructed and implemented in other ways and embodiments. In addition, where methods and steps described above indicate certain events occurring in a particular order, those of ordinary skill in the art having the benefit of this disclosure would recognize the ordering of certain steps may be modified and that such modifications are in accordance with the description herein. Additionally, certain steps may performed concurrently in a parallel process when possible, as well as performed sequentially as described above. Accordingly, the description herein should not be read as limiting such embodiments, as other embodiments also fall within the scope of this disclosure.

1. A cannula stabilization device, comprising:
a collar removably securable about a longitudinal axis of a cannula, the collar having a locking mechanism adapted to secure the collar about the cannula, wherein the locking mechanism further comprises an engaging member and a mateably engaging surface, and wherein the engaging member is interlockable with the engaging surface,
an anchoring mechanism including a plurality of legs extending downward from the collar in a substantially axial direction, each of the legs independently adjustable so that at least a portion of each leg extends in a substantially perpendicular direction from the collar,
wherein when positioned on a patient's skin, the collar and anchor mechanism stabilize the cannula from at least axial movement in a distal direction.

2. (canceled)

3. The device of claim 1, wherein the engaging member further comprises an arm having a protrusion extending substantially perpendicular from the arm, and the engaging surface further comprises a cavity, and wherein the protrusion of the engaging member is interlockable with the cavity of the engaging surface.

4. The device of claim 1, the collar further comprising a flange extending outwardly from each of two edges of the collar, each flange having a mateable opening, the locking mechanism further comprising a screw and nut, the screw extendable through the mated openings, and wherein tightening of the nut upon the screw secures the collar about the cannula.

5. The device of claim 1, the collar further comprising a flange extending outwardly from each of two edges of the collar, and the locking mechanism further comprising a clamp, the clamp positionable about each flange such that the clamp secure the collar about the cannula.

6-7. (canceled)

8. The device of claim 1, wherein each leg further comprises an adjustable height.

9. The device of claim 7, wherein each leg further comprises an adjustable angle of extension from the collar.

10. The device of claim 7, wherein the legs are removably adherable to the patient's skin.

11. The device of claim 6, wherein the anchoring mechanism further comprises a skirt substantially encircling the collar and extending substantially perpendicular to the collar.

12. The device of claim 6, the anchor mechanism further comprising a plate mountable on the patient's skin and having a channel therein, and the collar further comprising a distal portion adapted to slidingly engage the channel such that the cannula can be moved in a plane along the channel.

13. A device of claim 12, wherein the distal portion of the collar further comprises a skirt extending downward and outward from the collar.

14. A system comprising:
a cannula; and
a collar removably securable about a longitudinal axis of the cannula, the collar having a locking mechanism adapted to secure the collar about the cannula, wherein the locking mechanism further comprises an engaging member and a mateably engaging surface, and wherein the engaging member is interlockable with the engaging surface,
an anchoring mechanism including a plurality of legs extending downward from the collar in a substantially axial direction.
wherein when positioned on a patient's skin, the collar stabilizes the cannula form at least axial movement in a distal direction.

15. (canceled)

16. The system of claim 1, wherein each of the legs is independently adjustable so that at least a portion of each leg extends in a substantially perpendicular direction from the collar.

17. The system of claim 16, wherein the legs are removably adherable to the patient's skin.

18. A method for stabilizing a cannula at a surgical site comprising:
placing a desired portion of the cannula at the surgical site;
providing a cannula stabilization device comprising a collar having a locking mechanism adapted to removably secure the collar about the cannula, wherein the locking mechanism further comprises an engaging member and a mateably engaging surface, and wherein the engaging member is interlocked with the engaging surface;
securing the collar to the cannula with a plurality of legs extending downward from the collar in an axial direction toward a patient's skin.

19. The method of claim 18, further comprising:
anchoring the cannula stabilization device to the patient's skin.

20. The method of claim 19, wherein the anchoring the cannula stabilization device further comprises adjusting at least one of the plurality of legs so that at least a portion of the leg extends in a substantially perpendicular directicin from the collar onto the patient's skin.
